(19) United States
(12) Patent Application Publication
(10) Pub. No.: US 2010/0172972 A1
(43) Pub. Date: Jul. 8, 2010

(54) ENTERIC COATED PHARMACEUTICAL COMPOSITIONS

(75) Inventors: Veera Babu Taduri, Baroda (IN); Deepak Gondaliya, Baroda (IN); Mukti Pancham Singh Yadav, Baroda (IN)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: SUN PHARMACEUTICAL INDUSTRIES LIMITED, Mumbai (IN)

(21) Appl. No.: 12/601,437

(22) PCT Filed: May 21, 2008
(86) PCT No.: PCT/IN2008/000319

§ 371 Date: Mar. 22, 2010

(30) Foreign Application Priority Data
May 21, 2007 (IN) 950/MUM/2007

(51) Int. Cl. A61K 31/381; A61K 9/32; A61K 9/48

(52) U.S. Cl. 424/456; 514/438

(57) ABSTRACT

The present invention relates to an enteric coated pharmaceutical composition comprising a core in the from of pellets comprising a therapeutically effective amount of duloxetine or its pharmaceutically acceptable salt, the pellets having a size between 700 to 1000 μm; a separating layer surrounding the core, comprising one or more pharmaceutically acceptable film-forming polymers and pharmaceutically acceptable excipient(s), the separating layer being present in an amount ranging from about 5% to about 20% by weight of the composition, and an enteric layer surrounding the separating layer comprising about 8% to about 25% by weight of the composition of poly(methacrylic acid, ethyl acrylate) (1:1) neutralized to a pH of about 5.0, wherein the enteric coated pharmaceutical composition, when administered orally to human subjects on an empty stomach, provides a maximum plasma concentration of duloxetine ranging from about 25 ng/ml to about 45 ng/ml, occurring from about 5 to 7 hours.

### FIELD OF THE INVENTION

The present invention relates to pharmaceutical compositions of the anti-depressant drug, duloxetine, for oral administration, a process for preparing such formulations and a method of administering to a patient in need thereof.

### BACKGROUND OF THE INVENTION

Duloxetine is a selective serotonin and norepinephrine reuptake inhibitor (SSNRI), chemically described as (+)-N-methyl-3-(1-naphthalenyloxy)-3-(2-thienyl)propan-1-amine) with the molecular formula C₁₈H₁₉NOS. Duloxetine is a white to slightly brownish white solid and is slightly soluble in water. Although the exact mechanism of the antidepressant and central pain inhibitory action of duloxetine in humans is unknown, the antidepressant and pain inhibitory actions are believed to be because of its potentiation of serotonergic and noradrenergic activity in the central nervous system (CNS). Duloxetine is indicated for the treatment of major depressive disorder and for the treatment of diabetic peripheral neuropathic pain.

Duloxetine is commercially available in United States of America as delayed release capsules under the brand name Cymbalta®. The approved Cymbalta® delayed release capsules contain duloxetine hydrochloride equivalent to 20 mg, 30 mg and 60 mg of duloxetine. The inactive ingredients in the approved compositions include, hypromellose, hydroxypropyl methylcellulose acetate succinate, sodium lauryl sulfate, sucrose, sugar spheres, talc, titanium dioxide, FD&C Blue No. 2, iron oxide yellow and triethyl citrate.

Cymbalta® delayed release capsules contain enteric coated duloxetine pellets comprising a) a core consisting of duloxetine and a pharmaceutically excipient; b) optionally a separating layer; and c) an enteric layer comprising hydroxypropylmethylcellulose acetate succinate and a pharmaceutically acceptable excipient.

Duloxetine hydrochloride is acid labile, and acid hydrolysis of its ether linkage results in a thienyl alcohol and 1-naphthol. At pH 1.0, which is achieved under fasting conditions in vivo, 50% of drug in the dosage is hydrolyzed to 1-naphthol. At a pH of 2.0, 10% of the drug in the dosage degrades to 1-naphthol in one hour, and at a pH of 4.0, 10% degradation would take up to 63 hours. This acid sensitive compound has therefore been formulated as enteric coated dosage form to protect it from degradation.

Enteric coatings have been used for many years to delay the release of the drug from the dosage forms. Depending on the composition and/or thickness, the enteric coatings are resistant to acidic gastric fluids but are soluble at higher pH in the intestine. Therefore, enteric coated formulations do not release the drug in the acidic gastric fluids where the drug is susceptible to degradation. The enteric coating polymer may be selected from polymers soluble at pH existing in the upper part of the small intestine or in the latter part of the small intestine and accordingly the release of the drug is delayed by a time period required for the dosage form to transit to these parts of the intestine.

However, preparation of stable formulation is problematic if the enteric coating polymer itself leads to instability of the drug. Duloxetine has been found to react with many enteric coatings to form a slowly soluble or an insoluble coating. Further, duloxetine has been found to react with polymer degradation products or residual free acids present in the enteric polymers, hydoxypropylmethylcellulose acetate succinate (HPMCAS) and hydroxypropyl methyl cellulose phthalate (HPMCP) in dosage formulations to form succinamide and phthalamide impurities, respectively (J Pharm Sci. 1998, January; 87(1):p81-85). The rate of formation of these impurities is further accelerated by heat and humidity.

To overcome these disadvantages, a number of research endeavors have been directed towards preparing enteric coated pharmaceutical compositions of duloxetine.

U.S. Pat. No. 5,508,276(the '276 patent) discloses enteric coated pellets of duloxetine coated with hydroxypropylmethylcellulose acetate succinate as the enteric coating agent. This patent covers the Cymbalta® delayed release product and discloses HPMCAS as the compatible enteric coating agent.

United States application No. 20060165776 (the '776 application) discloses oral pharmaceutical compositions comprising a) a core comprising duloxetine or its pharmaceutically acceptable derivatives thereof and the said core comprises pharmaceutically inert nuclei and duloxetine mixed and compressed together; b) an intermediate layer comprising one or more polymers c) an enteric polymer comprising one or more enteric polymers; wherein the said composition is free of alkaline reacting compounds.

It has now been found that, enteric coated pharmaceutical compositions of duloxetine prepared using a neutralized methacrylic acid copolymer as the enteric polymer, are stable.

### SUMMARY OF THE INVENTION

In one aspect of the invention there is provided an enteric coated pharmaceutical composition comprising:
a) a core in the from of pellets comprising a therapeutically effective amount of duloxetine or its pharmaceutically acceptable salt, the pellets having a size between 700 to 1000 μm;
b) a separating layer surrounding the core, comprising one or more pharmaceutically acceptable film-forming polymers and pharmaceutically acceptable excipient(s), the separating layer being present in an amount ranging from about 5% to about 20% by weight of the composition; and
c) an enteric layer surrounding the separating layer comprising about 8% to about 25% by weight of the composition of poly(methacrylic acid, ethyl acrylate) (1:1) neutralized to a pH of about 5.0,
wherein the enteric coated pharmaceutical composition, when administered orally to human subjects on an empty stomach, provides a maximum plasma concentration of duloxetine ranging from about 25 ng/ml to about 45 ng/ml, occurring from about 5 to 7 hours.

### DETAILED DESCRIPTION OF THE INVENTION

The present invention provides an enteric coated pharmaceutical composition comprising:
a) a core in the from of pellets comprising a therapeutically effective amount of duloxetine or its pharmaceutically acceptable salt, the pellets having a size between 700 to 1000 μm;
b) a separating layer surrounding the core, comprising one or more pharmaceutically acceptable film-forming polymers and pharmaceutically acceptable excipient(s), the separating layer being present in an amount ranging from about 5% to about 20% by weight of the composition; and
c) an enteric layer surrounding the separating layer comprising about 8% to about 25% by weight of the composition of poly(methacrylic acid, ethyl acrylate) (1:1) neutralized to a pH of about 5.0,
wherein the enteric coated pharmaceutical composition, when administered orally to human subjects on an empty stomach, provides a maximum plasma concentration of duloxetine ranging from about 25 ng/ml to about 45 ng/ml, occurring from about 5 to 7 hours.

The core of the pharmaceutical composition may be a unitary core comprising therapeutically effective amount of duloxetine hydrochloride, or may be multiparticulate.

The term “multiparticulate” as used herein refers to plurality of units, each unit having, a size ranging from about 0.1 to about 1.5 mm diameter, and having a geometrical shape selected from granules, spheroids, beads, seeds and pellets.

The term “acrylic acid copolymer” as used herein, refers to a copolymer formed by copolymerization of monomers wherein at least one monomer is methacrylic acid and at least one monomer is ethylacrylate.

The term “neutralized” as used herein refers to a polymer which has been totally or partially neutralized by addition of suitable base.

“Cₘₐₓ” as used herein, means maximum plasma concentration of the duloxetine, produced by the ingestion of composition comprising duloxetine hydrochloride.

“Tₘₐₓ” as used herein, means time to the maximum observed plasma concentration (Cₘₐₓ) described above.

“AUC” as used herein, means area under the plasma concentration vs time curve, as calculated by the trapezoidal rule over the complete 24 hour interval for all the formulations.

“AUC₀₋ₜ” as used herein, means area under the plasma concentration vs time curve from 0 hours to the time (t) of last sample collected.

“AUC_{0-∞}” as used herein, means area under the plasma concentration vs time curve from 0 hours to infinity.

The term “therapeutically effective amount” as used herein refers to an amount or concentration of duloxetine which is effective in reducing, eliminating, treating, preventing or controlling one more symptoms of major depressive disorder or neuropathic pain associated with diabetic peripheral neuropathy.

According to the present invention, duloxetine as used herein can also be used in the form its pharmaceutically acceptable salt such as acid addition salts like hydrochloride, hydrobromide, sulfate, nitrate, and phosphate; or a salt with an organic acid selected from acetic, propanoic, hydroxyacetic, lactic, pyruvic, oxalic, malonic, succinic, maleic, fumaric, malic, cyclamic, salicyclic, p-aminosalicylic, and pamoic acid. The hydrochloride salt of duloxetine is the preferred salt used in the compositions of the present invention. The amount of duloxetine is in the range from about 1 mg to about 100 mg, more preferably in the range from about 10 mg to about 80 mg. Typically, the duloxetine is present in the amount ranging from about 1% to about 80% by weight of the composition. It may be noted that the amount of duloxetine or its pharmaceutically acceptable salts used in the compositions of the present invention are expressed in terms of % w/w of duloxetine.

According to one aspect of the present invention, the core is prepared by wet granulation technique, comprising the steps of blending duloxetine hydrochloride, mannitol, talc and optionally pharmaceutically inert excipients; granulating with a granulating fluid or solution or dispersion of binder; drying and sizing the granules. These granules can be used for further processing like coating with a separating layer, followed by coating with an enteric layer.

In one embodiment, the core is prepared by dry granulation technique, comprising the steps of blending duloxetine hydrochloride, hypromellose, mannitol, talc and optionally pharmaceutically inert excipients; dry granulating the blend by roller compactor or slugging; sizing the granules and then layering with a separating layer, followed by an enteric layer.

In another embodiment, the core is prepared by extrusion-spheronization technique, comprising the steps of blending duloxetine hydrochloride, hypromellose, mannitol, talc; adding water to the blend and extruding through a extruder; drying the extrudates and spheronizing the extrudates in a spheronizer; separating the granules or spheroids to select a desired size and then layering with a separating layer, followed by an enteric layer.

In yet another embodiment, the core is prepared by obtaining granules of duloxetine hydrochloride and suitable excipients; compressing them to obtain a unit core; coating the core with a separating layer, followed by coating with an enteric layer.

In a preferred embodiment, the core is prepared by coating inert non-pareil seeds with a layer comprising duloxetine or its pharmaceutically acceptable salt. Such inert non-pareil seeds are conventionally used in pharmaceutical science. The most preferred non-pareil seeds are prepared from starch and sucrose which is commonly employed in making pellets for pharmaceuticals. However, non-pareil seeds made of any pharmaceutically acceptable excipient may be used, including, for example microcrystalline cellulose, vegetable gums, waxes and the like. The size of non-pareil seeds may vary from about 0.1 mm to about 2 mm, preferably from about 700 to about 1000 μm. The non-pareil seeds may be present in amount ranging from about 10% to about 90% by weight of the composition.

The core comprising duloxetine or its pharmaceutically acceptable salts may be present in an amount ranging from about 40% to about 75% by weight of the composition.

The non-pareil seeds are coated with a solution or suspension comprising duloxetine or its pharmaceutically acceptable salt, binder, diluent, antiadherant, and optionally other excipients, in a suitable solvent or vehicle to form a drug layer. Examples of binders include, but are not limited to, hydroxypropyl methylcellulose, hydroxypropylcellulose, polyvinylpyrrolidone, methylcellulose, carboxymethylcellulose, acacia, gelatin and the like and mixtures thereof. The preferred binder is hydroxypropylmethylcellulose. The amount of binder ranges from about 1.0% to about 20%, preferably from about 5.0% to about 15% by weight of duloxetine.

Examples of diluent include but are not limited to mannitol, sorbitol, erythritol, microcrystalline cellulose, lactose, starch, magnesium carbonate and the like and mixtures thereof. The amount of diluent may be present in an amount from about 5% to about 50%, preferably from about 10% to about 30% by weight of duloxetine.

A particularly preferred drug layer comprises duloxetine hydrochloride, hypromellose 2910 (Methocel E 5 Premium), mannitol and talc. More preferably, the drug layer comprises about 10% to about 80% duloxetine hydrochloride, about 2% to about 20% hydroxypropyl methylcellulose 2910, about 5% to about 30% mannitol and about 1% to about 10% talc, wherein the percentages are by weight of the drug layer.

According to an embodiment of the present invention, there is a separating layer between the core containing duloxetine, and the enteric layer. The separating layer comprises one or more pharmaceutically acceptable film-forming polymers and pharmaceutically acceptable excipients(s). The separating layer when used, provides a smooth base for the application of the enteric layer, prolongs the resistance of the core to the acidic conditions, improves stability by minimizing the interaction between drug in the core and the enteric polymer in the enteric layer from coming into direct contact with each other; and also improves stability of drug from light exposure. The smoothing function of the separating layer is purely mechanical, the objective of which is to improve the coverage of the enteric layer and to avoid thin spots in it, caused by bumps and irregularities on the core. The separating layer may be present in an amount ranging from about 5% to about 20% by weight of the composition.

The separating layer of the present invention preferably comprises a sugar. It has been found that, when a pharmaceutically acceptable sugar is added to the separating layer, the core's resistance to acid conditions is markedly increased. Accordingly, such a sugar may be included in the separating layer applied to the core as dispersion or solution comprising a polymeric material and other excipients. A sugar, when included in the separating layer, reduces the quantity of enteric polymer required to obtain a given level of acid resistance. The sugar in the separating layer may be present in the amount ranging from about 1.0% to about 10% by weight of the composition.

In addition to sugar, the separating layer is composed of coherent or film forming polymeric materials and finely powdered solid excipients, which constitute fillers. For example, substances such as hydroxypropylmethylcellulose, hydroxypropylcellulose, polyvinylpyrrolidone, methylcellulose, carboxymethylcellulose, acacia, gelatin and the like may be used in small amounts to increase adherence and coherence of the separating layer. The film forming polymeric material in the separating layer may be present in the amount ranging from about 0.1 to about 10% by weight of the composition.

A particularly preferred separating layer comprises hypromellose 2910, sucrose and talc. More preferably, the separating layer comprises about 10% to about 70% hypromellose, about 10% to about 60% sucrose, and about 5% to about 40% talc, wherein the percentages are by weight of the separating layer.

According to an embodiment of the present invention, the enteric layer is present over the separating layer. The enteric layer contains a neutralized methacrylic acid copolymer which has been neutralized with a base to a pH of about 5.0. The methacrylic acid copolymers that can be used in the present invention include Poly(methacrylic acid, ethyl acrylate) where the ratio of free carboxyl groups to ester groups is 1:1. The suitable methacrylic acid copolymers include those which are marketed under the trade names, EUDRAGIT® L 30 D-55, EUDRAGIT L100-55 (Roehm GmbH), EASTACRYL 30D (Eastman chemical), KOLLICOAT® MAE 30 DP, KOLLICOAT® MAE 100P (BASF Fine chemicals), and the like. The methacrylic acid copolymer(s) may be used alone or in combination with other another enteric polymers to achieve the desired delayed release profile. The Poly(methacrylic acid, ethyl acrylate) (1:1) may be present in amount ranging from about 5% to about 50%, preferably from between 8% to about 25% by weight of the composition.

The enteric layer may be present in an amount ranging from about 15% to about 40% by weight of the composition. EUDRAGIT L 30 D-55 is an aqueous acrylic resin dispersion. It is an anionic copolymer derived from methacrylic acid and ethyl acrylate with a ratio of free carboxylic groups to the ester of approximately 1:1, and has a mean molecular weight of approximately 250,000. It is supplied as an aqueous dispersion containing 30% w/w of dry lacquer substance, and is marketed by Rohm Pharma Company, Germany.

KOLLICOAT® MAE 30 DP and KOLLICOAT® MAE 100 P are copolymers derived from methacylic acid and ethyl acrylate with a ratio of the components in the copolymer about 1:1. The KOLLICOAT® MAE grades have an anionic character with an average molecular weight of approximately 250,000. The Kollicoat® MAE grades are commercially available from BASF, Germany.

According to one embodiment, the base that is used to neutralize the enteric coating polymer may be selected from the group consisting of sodium hydroxide, potassium hydroxide, calcium hydroxide, magnesium hydroxide, aluminum hydroxide, ammonia, ammonium hydroxide, and mixtures thereof. The extent of neutralization may be carried out without adversely affecting the poly(methacrylic acid, ethyl acrylate) (1:1) properties. The poly(methacrylic acid, ethyl acrylate) (1:1) may be fully or partially neutralized with the base. Preferably, the base used for neutralization of poly(methacrylic acid, ethyl acrylate) (1:1) is sodium hydroxide and is neutralized to a pH of about 5.0. The sodium hydroxide may be used in amounts ranging from about 0.01% to about 10% by weight of the poly(methacrylic acid, ethyl acrylate) (1:1).

The enteric layer of the present invention may include a plasticizer, surfactant, pigments, anti-adherents, opacifying agents, colorants and the like, which are routinely employed in the preparation of coating solution or suspension. The plasticizers used in the present invention may be selected from polyethylene glycol 6000, triethyl citrate, triacetin, diethyl phthalate, tributyl sebacate and the like. The plasticizer may be present in an amount ranging from about 0.5% to about 10% by weight of the composition.

The anti-adherent is selected from group consisting of talc, magnesium stearate and silicon dioxide. The antiadherant may be present in an amount ranging from about 0.5% to about 15% by weight of the composition.

The opacifying agents may be selected from iron oxide, titanium dioxide and the like and mixtures thereof. The opacifying agent may be present from about 0.1% to about 10% by weight of the composition.

A particularly preferred enteric layer comprises EUDRAGIT®L 30 D-55 (30% aqueous dispersion), polyethyleneglycol 6000, talc, titanium dioxide, polysorbate 80 and sodium hydroxide. More preferably, the enteric layer comprises about 50% to about 70% EUDRAGIT®L30D55 (30% aqueous dispersion), about 1% to about 20% polyethyleneglycol 6000, about 5% to about 50% talc, about 0.01% to about 5% titanium dioxide, about 0.1% to about 10% polysorbate 80, and about 0.01% to about 5% sodium hydroxide, wherein the percentages are by weight of the enteric layer.

Another preferred enteric layer comprises KOLLICOAT® MAE 30DP (30% aqueous dispersion), triethylcitrate, talc and sodium hydroxide. More preferably the enteric layer comprises about 50% to about 70% KOLLICOAT® MAE 30DP, about 1% to about 20% triethylcitrate, about 5% to about 50% talc and about 0.001% to about 5% sodium hydroxide, wherein the percentages are by weight of the enteric layer.

According to an embodiment, the coated multiparticulates of the present invention can be filled into a hard gelatin capsule or optionally mixed with other pharmaceutically acceptable inert excipients and compressed into a tablet or minitablets, and these tablets may then be filled into hard gelatin capsules. Alternatively, the enteric coated pharmaceutical compositions of the present invention may be provided in the form of tablets.

In an embodiment, the enteric coated pharmaceutical compositions of duloxetine hydrochloride of the present invention when administered orally to human subjects on an empty stomach, provides a maximum plasma concentration of duloxetine or its pharmaceutically acceptable salt ranging from about 25 ng/ml to about 45 ng/ml occurring from about 5 to 7 hours.

In an embodiment, the enteric coated pharmaceutical compositions of duloxetine hydrochloride of the present invention are bioequivalent to duloxetine hydrochloride delayed release capsules marketed under the trade name, Cymbalta® by Eli Lilly, USA.

The term “bioequivalent” as used herein means that the 90% confidence interval of the relative means of Cₘₐₓ, AUC₀₋ₜ and AUC_{0-∞} of the enteric coated pharmaceutical compositions of the present invention to the relative mean of Cₘₐₓ, AUG₀₋ₜ and AUC_{0-∞} of duloxetine delayed release compositions of equivalent dose, sold under the trade name Cymbalta®, are within 80-125%.

Enteric coated, multiparticulate pharmaceutical compositions of the present invention may be prepared according to the following process:
a) A dispersion of duloxetine hydrochloride was prepared by homogenizing duloxetine hydrochloride, hypromellose, mannitol and talc in water.
b) The homogenized drug dispersion was sprayed on to the sugar spheres in a fluidized bed processor.
c) The pellets were dried till moisture content was less than 1% w/w.
d) The intermediate coating solution was prepared by dispersing talc in aqueous solution of hypromellose and sucrose.
e) The solution obtained in step d, was sprayed over the drug pellets obtained in step c.
f) The enteric coating solution was prepared by adding a dilute solution of sodium hydroxide to the aqueous dispersion of methacrylic acid copolymer followed by addition of aqueous solution of polyethylene glycol, titanium dioxide, ploysorbate 80 and talc dispersion, wherein the Ph of the enteric coating solution was kept at pH 5.0.
g) The enteric coating was applied over pellets obtained in step e.
h) The enteric coated pellets obtained were filled into hard gelatin capsules.

It will be understood by those of skill in the art that numerous modifications can be made without departing from the spirit of the present invention. Therefore, it should be clearly understood that the following examples are illustrative only and should not to be construed to limit the scope of the present invention.

### Comparative Example 1

Enteric coated pharmaceutical compositions of Duloxetine hydrochloride were prepared as shown in Table 1.

TABLE 1

| | | |
|---|---|---|
| Ingredients | Quantity (mg/cap) | % w/w |

Core pellets

| | | |
|---|---|---|
| Sugar spheres | 124.81 | 33.22 |
| Duloxetine Hydrochloride | 67.30 | 17.92 |
| Hypromellose 2910 | 6.73 | 1.79 |
| Mannitol | 13.46 | 3.58 |
| Talc | 3.37 | 0.89 |

Separating layer

| | | |
|---|---|---|
| Hypromellose 2910 | 19.17 | 5.10 |
| Sucrose | 14.38 | 3.83 |
| Talc | 9.57 | 2.55 |

Enteric layer

| | | |
|---|---|---|
| EUDRAGIT L 30D-55 (on dry | 80.00 | 21.30 |
| solid content basis) | | |
| Polyethylene glycol 6000 | 7.81 | 2.08 |
| Talc | 24.64 | 6.56 |
| Titanium dioxide | 0.87 | 0.23 |
| Polysorbate 80 | 3.55 | 0.95 |
| Total | 375.66 | 100.00 |

### Process of Manufacture:

A homogenized dispersion of Duloxetine hydrochloride was prepared in aqueous solution of hypromellose and then mannitol, talc were added to the dispersion. The homogenized drug dispersion was then sprayed on to the sugar spheres (20/25 mesh) in fluidized bed equipment. The drug pellets were dried till the moisture content was less than 1.0% w/w. The separating layer coating solution was prepared by dispersing talc in an aqueous solution of hypromellose and sucrose. This solution was applied over drug pellets in Fluid bed equipment (FBE) to form the separating layer. A coating solution was prepared by mixing aqueous solution of polyethylene glycol in methacrylic acid copolymer dispersion and then adding talc dispersion to the same. An enteric layer was applied over these coated pellets. The enteric coated pellets were dried in FBE till moisture was not more than 1.0% w/w. The enteric coated pellets were then filled in hard gelatin capsules.

An in vitro dissolution test was conducted for duloxetine hydrochloride delayed release capsule compositions of comparative Example 1. The dissolution study was performed using USP Type 1 dissolution apparatus, operating at 37° C. with a basket rotating at a speed of 50 rpm. The capsules were tested in 900 ml, 0.1N hydrochloride for first 120 minutes, followed by 900 ml of pH 6.8 phosphate buffer for 90 minutes. The results are shown in Table 2.

TABLE 2
Dissolution profile of Duloxetine hydrochloride delayed
release capsules of comparative Example 1.

| | Cumulative percentage (%) release of |
|---|---|
| Time (minute) | duloxetine hydrochloride |
| 120 | 3.46 |
| 15 | 3.51 |
| 30 | 18.38 |
| 45 | 32.74 |
| 60 | 47.72 |
| 90 | 61.96 |

The duloxetine hydrochloride capsules were analyzed using High Performance Liquid Chromatography (HPLC) for the presence and the amount of duloxetine hydrochloride impurities. The results are shown in Table 3.

TABLE 3

| Type of Impurity | Percent (%) |
|---|---|
| Single Maximum impurity | 0.14 |
| Total Impurity | 0.26 |

It is clearly evident from Table 2 above that a maximum release of duloxetine was only about 60%. Also, Table 3 indicates that a total impurity of 0.26% was formed.

The compositions prepared as per comparative Example 1, were charged at 60° C. for 3 days. The results of dissolution study at the end of 3 days are summarized in Table 4. The duloxetine hydrochloride capsules were analyzed using High Performance Liquid Chromatography (HPLC) for the presence and the amount of duloxetine hydrochloride impurities. The results are summarized in Table 5.

TABLE 4

| | Cumulative percentage (%) of |
|---|---|
| | duloxetine hydrochloride |
| Time (minutes) | Comparative Example 1 |
| 120 | 8.46 |
| 15 | 2.42 |
| 30 | 7.76 |
| 45 | 21.55 |
| 60 | 33.50 |
| 90 | 51.34 |

TABLE 5

| Type of Impurity | Percent (%) |
|---|---|
| Single Maximum impurity | 0.66 |
| Total Impurity | 1.16 |

It may be noted from Tables 4 and Table 5 above, that at the end of 3 days at 60° C., the duloxetine delayed release capsules were found to release only about 50% of duloxetine, and the total impurities in the duloxetine delayed release capsules had gone up to 1.16%

### Example 1

Enteric coated pharmaceutical compositions of duloxetine hydrochloride of the present invention which are coated with neutralized methacrylic acid copolymer dispersion are shown in Table 6.

TABLE 6

| | | |
|---|---|---|
| Ingredients | Quantity (mg/cap) | % w/w |

Core pellets

| | | |
|---|---|---|
| Sugar spheres | 124.81 | 33.18 |
| Duloxetine Hydrochloride | 67.30 | 17.89 |
| Hypromellose 2910 | 6.73 | 1.79 |
| Mannitol | 13.46 | 3.58 |
| Talc | 3.37 | 0.89 |

Separating layer

| | | |
|---|---|---|
| Hypromellose 2910 | 19.17 | 5.09 |
| Sucrose | 14.38 | 3.82 |
| Talc | 9.57 | 2.54 |

Enteric layer

| | | |
|---|---|---|
| EUDRAGIT L 30D-55 (on dry | 80.00 | 21.27 |
| solid content basis) | | |
| Polyethylene glycol 6000 | 7.81 | 2.08 |
| Talc | 24.64 | 6.55 |
| Titanium dioxide | 0.87 | 0.23 |
| Polysorbate 80 | 3.55 | 0.94 |
| Sodium Hydroxide | 0.51 | 0.13 |
| Total | 376.17 | 100.00 |

### Process of Manufacture:

A homogenized dispersion of Duloxetine hydrochloride was prepared in an aqueous solution of hypromellose and then mannitol and talc were added to the dispersion. The homogenized drug dispersion was sprayed on to the sugar spheres (20/25 mesh) in fluidized bed equipment. The drug pellets were dried till the moisture content was less than 1.0% w/w. A coating solution was prepared by dispersing talc in an aqueous solution of hypromellose and sucrose. The solution was applied over drug pellets in the Fluid bed equipment (FBE) to form the separating layer. A second coating solution was prepared by adding a dilute solution of sodium hydroxide to an aqueous dispersion of methacrylic acid copolymer followed by addition of aqueous solution of polyethylene glycol and talc dispersion. The pH of the enteric coating solution thus obtained was maintained at pH 5.0. This was then used to form the enteric layer. The enteric coated pellets were dried in FBE till moisture content was not more than 1.0% w/w. The enteric coated pellets were then filled in hard gelatin capsules.

### In Vitro Dissolution Study

The in vitro dissolution tests were conducted for duloxetine hydrochloride delayed release capsule compositions of Example 1. The dissolution study was performed using USP Type 1 dissolution apparatus, operating at 37° C. with the basket rotating at a speed of 50 rpm. The capsules were tested in 900 ml, 0.1N hydrochloride for first 120 minutes, followed by 900 ml of pH 6.8 phosphate buffer for 90 minutes. The results are shown in Table 7.

TABLE 7
Dissolution profile of Duloxetine hydrochloride
delayed release capsules of Example 1

| | Cumulative percentage (%) release of |
|---|---|
| Time (minute) | duloxetine hydrochloride |
| 120 | 2.05 |
| 15 | 34.08 |
| 30 | 86.15 |
| 45 | 93.04 |
| 60 | 96.28 |
| 90 | 97.45 |

### Stability Study

The duloxetine hydrochloride capsules having the compositions of Example 1 were packed in High Density Polyethylene (HDPE) bottles and stored at 25° C./60% Relative Humidity, 30° C./65% Relative Humidity and 45° C./75% Relative Humidity for a period of about 3 months The capsules were analyzed using High Performance Liquid Chromatography (HPLC) for assay, percent drug release and the amount of total duloxetine hydrochloride impurities. The results obtained are shown in Table 8.

TABLE 8

| | | Dissolution | | | |
|---|---|---|---|---|---|
| | | Acid | Buffer (PH 6.8, | | Degradation |
| Storage | Time | (0.1 N HCl, | phosphate buffer, | | Total |
| Condition | points | After 120 min) | After 90 min) | Assay | impurities (%) |
| | Initial | 1.86 | 91.43 | 100.51 | 0.101 |
| 25° C./60% RH | 3 month | -Nil- | 93.54 | 96.31 | 0.097 |
| 30° C./65% RH | 2 month | 4.93 | 84.26 | 96.70 | 0.089 |
| | 3 month | 3.92 | 90.69 | 96.56 | 0.099 |
| 40° C./75% RH | 1 month | 8.65 | 88.97 | 90.64 | 0.105 |
| | 2 month | 8.92 | 91.16 | 94.52 | 0.126 |
| | 3 month | 1.10 | 91.85 | 97.26 | 0.122 |

The dissolution study as shown in Table 7 showed a complete release of drug when pellets were coated with neutralized methacrylic acid copolymer, suggesting that neutralization of free carboxylic acid groups present in the polymer is necessary to restrict the interaction between free acid groups and drug. From the Table 8 it is clearly evident that the degradation was also found to be significantly less in comparison to compositions of comparative Example 1.

### Bioavailability Study

The bioavailability study of duloxetine hydrochloride delayed release capsule (60 mg) composition of Example 1 was carried out on healthy human male volunteers. The study was conducted on empty stomach and Non-empty stomach conditions. The results are shown in the Table 9 and Table 10.

TABLE 9
Pharmacokinetic parameters obtained through the bioavailability
studies of duloxetine hydrochloride delayed release capsules
of Example 1 on empty stomach condition.

| | Pharmacokinetic parameter | | | |
|---|---|---|---|---|
| | Ln Cₘₐₓ | Ln AUC₀₋ₜ | Ln AUC_{0-∞} | |
| | (ng/ml) | (ng · hr/ml) | (ng · mg/ml) | Tₘₐₓ (hr) |
| Duloxetine Delayed | 29.42 | 507.16 | 561.68 | 7.00 |
| release capsule | | | | |
| (Example 1) | | | | |

[n = 18]

TABLE 10
Pharmacokinetic parameters obtained through the bioavailability
studies of duloxetine hydrochloride delayed release capsules
of Example on Non-empty stomach condition.

| | Pharmacokinetic parameter | | | |
|---|---|---|---|---|
| | Ln Cₘₐₓ | Ln AUC₀₋ₜ | Ln AUC_{0-∞} | |
| | (ng/ml) | (ng · hr/ml) | (ng · mg/ml) | Tₘₐₓ (hr) |
| Duloxetine Delayed | 33.73 | 588.26 | 631.74 | 9.00 |
| release capsule | | | | |
| (Example 1) | | | | |

[n = 17]

### Example 3

The compositions of the present invention prepared as per Example 1 were charged at 60° C. for 3 days. The in vitro dissolution tests were conducted using USP Type 1 dissolution apparatus, operating at 37° C. with the basket rotating at a speed of 50 rpm. The capsules were tested in 900 ml, 0.1N hydrochloride for first 120 minutes, followed by 900 ml of pH 6.8 phosphate buffer for 90 minutes. The results of dissolution study obtained are summarized in Table 11.

TABLE 11

| | Cumulative percentage (%) release of |
|---|---|
| | duloxetine hydrochloride |
| Time (minutes) | Composition of Example 1 |
| 120 | 6.23 |
| 15 | 40.41 |
| 30 | 93.25 |
| 45 | 97.26 |
| 60 | 97.87 |
| 90 | 98.94 |

The duloxetine hydrochloride capsules were analyzed using High Performance Liquid Chromatography (HPLC) for the presence and the amount of duloxetine hydrochloride impurities. The results obtained are shown in Table 12.

TABLE 12

| Type of Impurity | Percent (%) |
|---|---|
| Single Maximum impurity | 0.04 |
| Total Impurity | 0.11 |

### Example 4

Enteric coated pharmaceutical compositions of duloxetine prepared according to the present invention are described in the table below.

| | Quantity | | | |
|---|---|---|---|---|
| | mg/cap | | | |
| Ingredients | 20 mg | 30 mg | 60 mg | % w/w |

Core pellets

| | | | | |
|---|---|---|---|---|
| Sugar spheres (20/25 mesh) | 41.53 | 62.29 | 124.59 | 39.25 |
| Duloxetine Hydrochloride | 22.45 | 33.68 | 67.35 | 21.22 |
| Hypromellose 2910 | 2.24 | 3.36 | 6.72 | 2.12 |
| (Methocel E 5 Premium) | | | | |
| Mannitol | 4.48 | 6.71 | 13.44 | 4.23 |
| Talc | 1.12 | 1.68 | 3.36 | 1.06 |

Separating layer

| | | | | |
|---|---|---|---|---|
| Hypromellose 2910 | 6.374 | 9.56 | 19.12 | 6.02 |
| Sucrose | 4.80 | 7.20 | 14.40 | 4.54 |
| Talc | 3.18 | 4.77 | 9.55 | 3.01 |

Enteric layer

| | | | | |
|---|---|---|---|---|
| Methacrylic acid copolymer | 12.27 | 18.411 | 36.82 | 11.60 |
| (KOLLICOAT ® MAE 30 DP) | | | | |
| Triethyl citrate | 2.45 | 3.68 | 7.36 | 2.32 |
| Talc | 4.91 | 7.36 | 14.73 | 4.64 |
| Sodium hydroxide | 0.003 | 0.004 | 0.008 | 0.0025 |
| Total | 105.80 | 158.73 | 317.46 | 100.00 |

### In Vitro Dissolution Study

In vitro dissolution study was conducted for duloxetine hydrochloride delayed release capsules of the present invention as shown in Example 4 (60 mg) and compared with the marketed product of duloxetine delayed release capsules (Cymbalta®) by Eli lily. The study was performed using USP Type 1 dissolution apparatus, operating at 37° C. with the basket rotating at a speed of 100 rpm. The capsules were tested in 1000 ml, 0.1N hydrochloride for first 120 minutes, followed by 1000 ml of pH 6.8 phosphate buffer for 90 minutes. The results are summarized in Table 13.

TABLE 13
Comparative dissolution profile of duloxetine delayed
release capsules (60 mg) of Example 4 and Cymbalta ® delayed
release capsules, 60 mg.

Cumulative percent (%) drug release

| Time | Compositions of Example 4 | Cymbalta ® delayed |
|---|---|---|
| (min) | (60 mg) | release capsules (60 mg) |
| 120 | 0.45 | — |
| 10 | 7.16 | 24.84 |
| 20 | 73.68 | 64.48 |
| 30 | 89.08 | 79.37 |
| 45 | 92.63 | 90.61 |
| 60 | 94.32 | 95.94 |
| 90 | 95.21 | 98.86 |

From the above study, it is clearly evident that the in vitro release profile of duloxetine delayed release capsules of the present invention are comparable to that of the commercially available product (Cymbalta® capsules).

### Stability Study

The duloxetine hydrochloride capsules having the compositions of Example 4 (60 mg) were packed in Polyvinyl chloride (PVC)/Polyvinylidine Chloride (PVDC) and High Density Polyethylene (HDPE) bottles and stored at 30° C./65% Relative Humidity and 45° C./75% Relative Humidity for a period of about 1 month. The capsules were analyzed using High Performance Liquid Chromatography (HPLC) for assay, percent drug release and the amount of total duloxetine hydrochloride impurities. The results are shown below:

### [a] PVC/PVDC Packing

| | | Dissolution | | | |
|---|---|---|---|---|---|
| | | Acid | Buffer (PH 6.8, | | Degradation |
| Storage | Time | (0.1 N HCl, | phosphate buffer, | | Total |
| Condition | points | After 120 min) | After 90 min) | Assay | impurities (%) |
| | Initial | 0.48 | 95.21 | 99.78 | 0.070 |
| 30° C./65% RH | 1 month | 1.75 | 93.05 | 105.23 | 0.192 |
| 40° C./% RH | 1 month | 1.81 | 93.01 | 105.23 | 0.170 |

### [b] HDPE Bottle

| | | Dissolution | | | |
|---|---|---|---|---|---|
| | | Acid | Buffer (PH 6.8, | | Degradation |
| Storage | Time | (0.1 N HCl, | phosphate buffer, | | Total |
| Condition | points | After 120 min) | After 90 min) | Assay | impurities (%) |
| | Initial | 0.48 | 95.21 | 99.78 | 0.070 |
| 30° C./65% RH | 1 month | 2.35 | 92.31 | 104.69 | 0.153 |
| 40° C./% RH | 1 month | 2.00 | 97.49 | 104.58 | 0.135 |

### Example 6

The bioavailability study of duloxetine hydrochloride delayed release capsule (60 mg) composition of Example 4 was carried out on healthy human male volunteers (n=15) on empty stomach condition. The results are shown in Table 14.

TABLE 14
Pharmacokinetic parameters obtained through the bioavailability
study of duloxetine hydrochloride delayed release capsules
of Example 4 on empty stomach condition.

| | Pharmacokinetic parameter | | | |
|---|---|---|---|---|
| | Ln Cₘₐₓ | Ln AUC₀₋ₜ | Ln AUC_{0-∞} | |
| | (ng/ml) | (ng · hr/ml) | (ng · mg/ml) | Tₘₐₓ (hr) |
| Duloxetine Delayed | 32.40 | 551.73 | 627.1 | 6.00 |
| release capsule | | | | |
| (Example 4) | | | | |

The invention having been described, it will be readily apparent to those skilled in the art that further changes and modifications in actual implementation of the concepts and embodiments described herein can easily be made or may be learned by practice of the invention, without departing from the spirit and scope of the invention as defined by the following claims.

We claim:

1. An enteric coated pharmaceutical composition comprising
a. a core in the from of pellets comprising a therapeutically effective amount of duloxetine or its pharmaceutically acceptable salt, the pellets having a size between 700 to 1000 μm;
b. a separating layer surrounding the core, comprising one or more pharmaceutically acceptable film-forming polymers and pharmaceutically acceptable excipient(s), the separating layer being present in an amount ranging from about 5% to about 20% by weight of the composition, and
c. an enteric layer surrounding the separating layer comprising about 8% to about 25% by weight of the composition of poly(methacrylic acid, ethyl acrylate) (1:1) neutralized to a pH of about 5.0,
wherein the enteric coated pharmaceutical composition, when administered orally to human subjects on an empty stomach, provides a maximum plasma concentration of duloxetine ranging from about 25 ng/ml to about 45 ng/ml, occurring from about 5 to 7 hours.

2. An enteric coated pharmaceutical composition as claimed in claim 1, wherein the poly(methacrylic acid, ethyl acrylate) is neutralized with a base selected from the group comprising sodium hydroxide, potassium hydroxide, calcium hydroxide, magnesium hydroxide, aluminum hydroxide, ammonia and ammonium hydroxide.

3. An enteric coated pharmaceutical composition as in claim 2, wherein the base is used in an amount ranging from about 0.01% w/w to about 10% w/w of the poly(methacrylic acid, ethyl acrylate).

4. An enteric coated pharmaceutical composition as in claim 1, wherein the pharmaceutically acceptable excipient present in the separating layer is a sugar.

5. An enteric coated pharmaceutical composition as in claim 4, wherein the sugar is sucrose.
